(19) United States
(12) Patent Application Publication
(10) Pub. No.: US 2010/0049205 A1
(43) Pub. Date: Feb. 25, 2010

(54) SURGICAL INSTRUMENT FOR STIMULATING, IN THE INTRAOPERATIVE PHASE, THE FUNCTIONING INSTABILITY OF ACETABULAR COMPONENTS OF HIP PROSTHESES

(75) Inventors: Roberto Giacometti, Milan (IT); Manuela Teresa Raimondi, Milano (IT); Ariana Colombini, Mesero (Milan) (IT)

Correspondence Address: HEDMAN & COSTIGAN P.C., 1185 AVENUE OF THE AMERICAS, NEW YORK, NY, 10036, US

(73) Assignee: Istituto Ortopedico Galeazzi S.P.A, Milan (IT)

(21) Appl. No.: 12/448,341

(22) PCT Filed: Dec. 21, 2007
(86) PCT No.: PCT/IB2007/004128

§ 371 Date: Aug. 24, 2009

(30) Foreign Application Priority Data
Dec. 28, 2006 (IT) MI2006A002533

(51) Int. Cl. A61B 17/58

(52) U.S. Cl. 606/99

(57) ABSTRACT

A surgical instrument (10) is described for the implanting of acetabular components of hip prostheses. The instrument comprises a main stem (12) which acts as a dynamometric impactor for obtaining the implantation of the acetabular component and which comprises at least one torsional dynamometer (14), coaxial to the main stem (12), for the application and measuring of a torsional moment on the acetabular component implanted. The instrument also comprises a side mechanism (16), which includes a secondary stem (18), parallel to the main stem (12), a handle (20) and at least one axial dynamometer (22), orthogonal to the stem (18), which connects the secondary stem (18) to the handle (20) for the application and measuring of a destabilizing force on the acetabular component implanted.

The present invention relates to the biomedical sector. More specifically, the present invention relates to a surgical instrument for the implanting of hip prostheses, in particular non-cemented acetabular components (metal-backs press-fit inserted in the acetabular cavity).

Hip prostheses are the most widely used joint endoprostheses in reconstructive orthopedic surgery. A hip prosthesis normally consists of a stem made of metal, fixed in the diaphysis channel of the thigh-bone, a femoral head made of metal or ceramics, connected to the stem by means of a conical coupling, an acetabular cup which is articulated on the femoral head, made of UHMWPE (ultra high molecular weight polyethylene) or, more rarely, of ceramics or metal, and a metallic acetabular shell or “metal-back”, which rigidly envelops the acetabular cup and which must be inserted in the acetabular cavity of the patient's pelvis.

In order to obtain so-called “press-fit” implants of the acetabular component or shell in the relative cavity, orthopedic surgeons currently use an instrument, called impactor, which allows this acetabular component to be positioned in its cavity and to be implanted by a force of impact.

The clinical duration of an acetabular component implanted in this way or, in other words, not cemented or equipped with transacetabular screws, is strictly linked to the post-operative stability of the implant at the interface with the bone. During the arthroplastic intervention of the hip, in fact, it is difficult to objectively establish the stability of a metal-back press-fit inserted in the acetabular cavity.

Using an impactor of the traditional type, an orthopedic surgeon is capable of implanting an acetabular component relying on his own sensitivity and experience. The tests he effects on the implant are therefore subjective and do not envisage the simulation of a loading condition similar to that in vivo. The necessity is therefore evident of envisaging an instrument which allows the surgeon to reproduce, quantitatively and in the intra-operative phase, the mechanical stress which acts on the metal-back of a hip prosthesis in vivo.

An objective of the present invention is therefore to provide a surgical instrument for the implantation of hip prostheses, in particular of non-cemented acetabular components, which envisages the integration, on a single instrument, of both an implantation system and also a system for the simulation of the acetabular biomechanics in the intra-operative phase.

A further objective of the present invention is to provide a surgical instrument for the implantation of hip prostheses which allows the loading condition which acts in vivo on the implant, to be reproduced, quantitatively and in the intra-operative phase.

Another objective of the present invention is to provide a surgical instrument for the implantation of hip prostheses which can be re-used various times, with the possibility of sterilization in an autoclave, for example.

Yet another objective of the present invention is to provide a surgical instrument for the implantation of hip prostheses which is simple and particularly economical to produce.

These and other objectives according to the present invention are achieved by providing a surgical instrument for the implantation of hip prostheses, in particular non-cemented acetabular components, as specified in claim 1.

Further characteristics of the invention are indicated in the subsequent claims.

The characteristics and advantages of a surgical instrument for the implantation of hip prostheses according to the present invention will appear more evident from the following illustrative and non-limiting description, referring to the enclosed schematic drawings, in which:

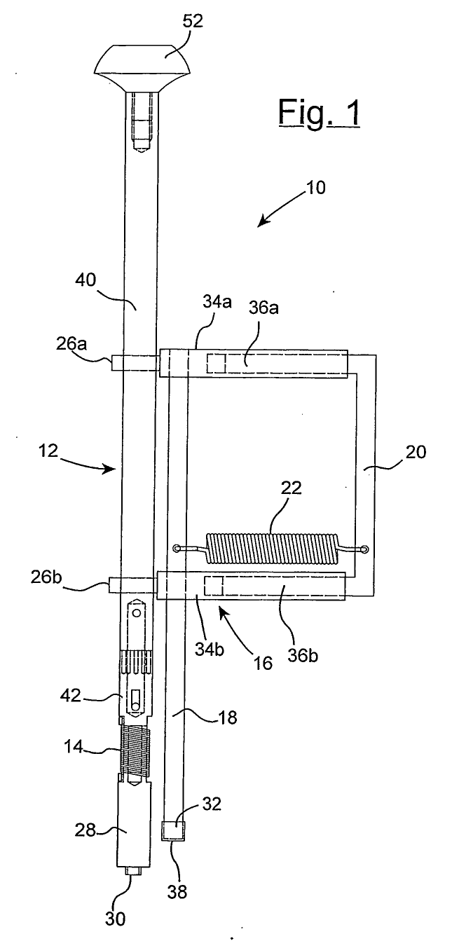

FIG. 1 is an overall partially sectional view of a surgical instrument for the implantation of hip prostheses according to the present invention;

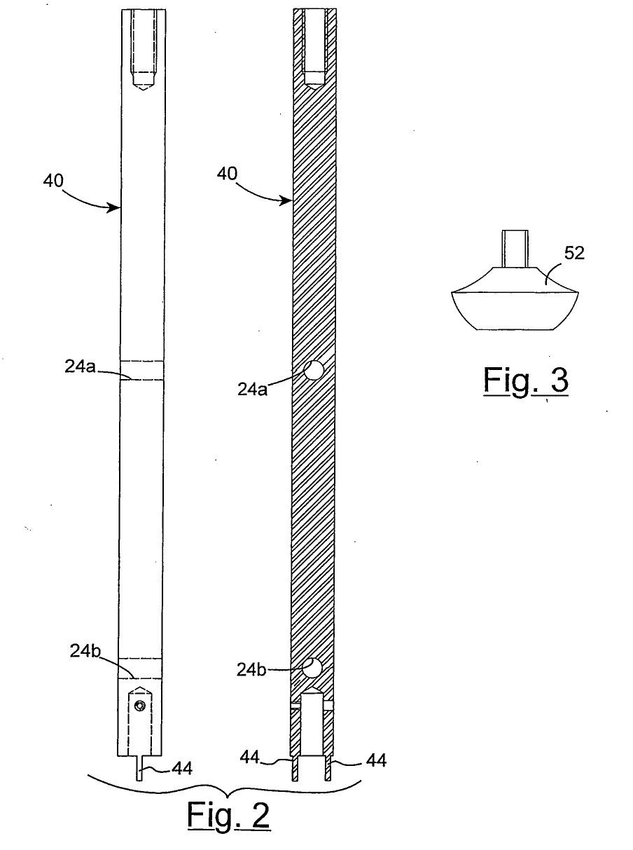

FIG. 2 shows two side views, obtained along two different sectional planes, of part of the main stem of the instrument of FIG. 1;

FIG. 3 is a raised side view of the impact component of the instrument of FIG. 1;

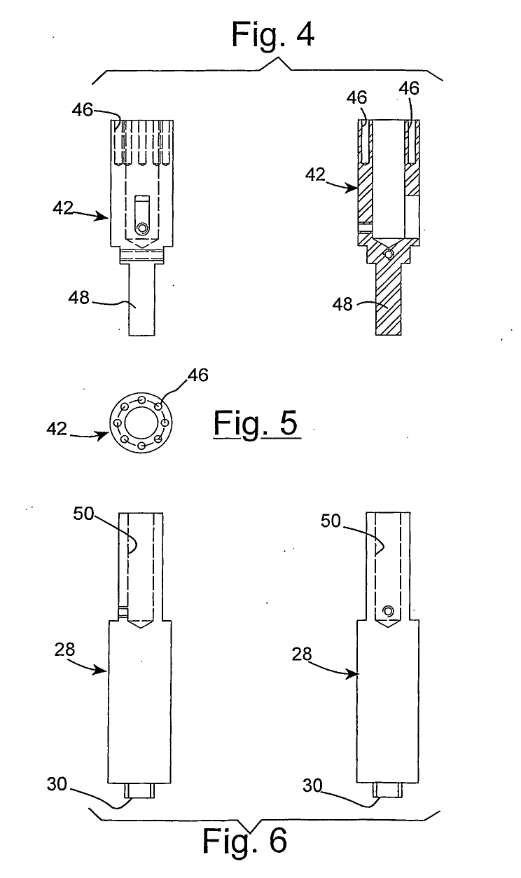

FIG. 4 shows two raised side views, obtained along two different sectional planes, of the central element of the main stem of the instrument of FIG. 1;

FIG. 5 is a view from above of the central element shown in FIG. 4;

FIG. 6 shows two raised side views, obtained along two different sectional planes, of the lower element of the main stem of the instrument of FIG. 1;

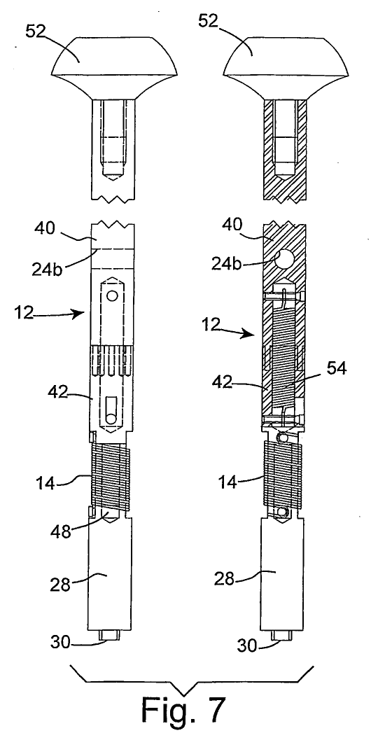

FIG. 7 shows two raised side views, obtained along two different sectional planes, of the main stem of the instrument of FIG. 1, equipped with the impact component of FIG. 3; and

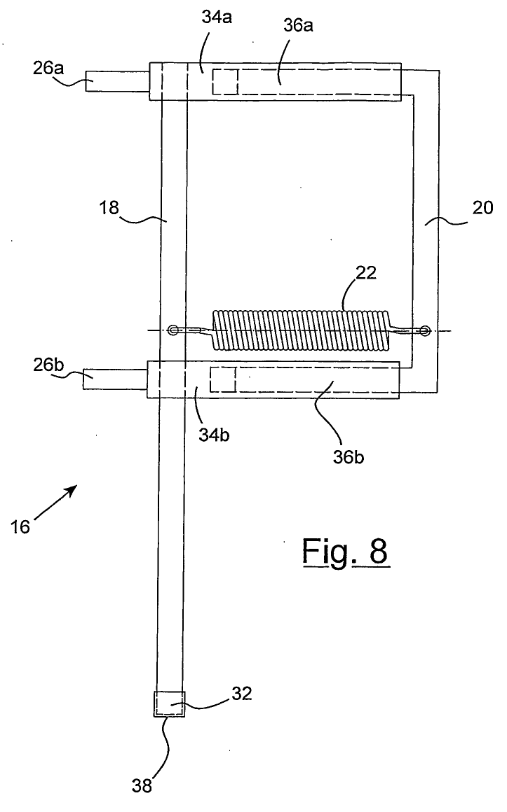

FIG. 8 is a raised partially sectional side view of the side mechanism of the instrument of FIG. 1.

With reference to the figures, these show a surgical instrument for the implanting of hip prostheses according to the present invention, indicated as a whole with the reference number 10.

The surgical instrument 10 in its whole, substantially consists of two fundamental parts:
a main stem 12, which acts as a dynamometric impactor for obtaining the implantation of a generic acetabular component (not shown), comprising at least one torsional dynamometer 14 coaxial thereto for the application and measurement of a torsional moment on the acetabular component implanted, also called metal-back and having the form of a dome or spherical cap; and
a side mechanism, indicated as a whole with the reference number 16 (FIG. 8), comprising a secondary stem 18, parallel to the main stem 12, a handle 20 and at least one axial dynamometer 22, orthogonal to the stem 18, for the application and measurement of a destabilizing force on the acetabular component implanted.

The destabilizing force reproduces the action of the articular load, whereas the torsional moment represents the friction moment which acts in vivo. The two stress actions consequently allow the condition in which the patient loads the prosthesis, to be simulated in the intra-operative phase.

The two parts 12 and 16 of the instrument 10 are connected with each other by means of a pair of pass-through cylindrical holes 24a and 24b, situated on the main stem 12 and have an axis perpendicular to the axis of the stem 12 itself. Two pins 26a and 26b obtained on the side mechanism 16 are respectively inserted, by sliding, inside the holes 24a and 24b.

On the free end above the main stem 12, there is an impact component 52, suitable for receiving the impact force to be transmitted to the acetabular component of the prosthesis. In correspondence with the lower free end of the main stem 12, on the other hand, a lower element 28 is applied (FIG. 6), which can be rotated, equipped with a threaded terminal end 30 for connection with the hole normally situated in correspondence with the top of the metal-back. The secondary stem 18 is in turn equipped with a lower free end 32, preferably covered with a protective hood 38, which, together with the terminal end 30 of the lower element 28, forms the interface system with the metal-back.

The secondary stem 18, connected to the handle 20, is capable of sliding parallel to the main stem 12 until coming to a stop against the internal edge of the metal-back. The adaptability of the interface system with the metal-back of the surgical instrument 10 with acetabular shells having different sizes, is in fact guaranteed by the possibility of sliding the secondary stem 18 with respect to the main stem 12. The only expedient to be adopted relates to the dimensioning of the two stems: the sum of the radius of the main stem 12 and the diameter of the secondary stem 18 must not be greater than the internal radius of the metal-back.

Surgical compatibility is ensured if the side handle 20 is positioned at a suitable height from the threaded terminal end 30 of the element 28, as the side mechanism 16 must remain external with respect to the patient's pelvis on the operating table.

The secondary stem 18 is made integral with a pair of sleeves 34a and 34b, in turn integral and coaxial with the pins 26a and 26b, respectively. The two end portions 36a and 36b of the side handle 20 are able to slide in the two sleeves 34a and 34b, whereas the axial dynamometer 22, which connects the secondary stem 18 and the side handle 20, consists of a pull spring.

The side handle 20 allows the application of a traction force in a perpendicular direction with respect to the main stem 12 and secondary stem 18. The spring 22 transmits this force to the secondary stem 18 which, in turn, exerts it on the internal edge of the metal-back against which it is buffered by means of its lower end 32. The same spring 22, suitably calibrated, allows the destabilizing force applied on the metal-back to be measured, consequently operating as an axial dynamometer.

A further system allows the direction of the destabilizing force inside the acetabular cavity to be selected. Between the lower element 28 and the upper tubular body 40 (FIG. 2), i.e. that in which the side mechanism 16 is inserted, of the main stem 12, there is in fact a central element 42 (FIG. 4) coaxial to this.

In correspondence with the reciprocal interface portions, the upper body 40 is equipped with a pair of pins 44, whereas the central element 42 has a plurality of axial holes 46 arranged along a circumference. The upper body 40 of the main stem 12 can therefore be press-fit connected with the central element 42 according to various rotation degrees, by insertion of the pins 44 in the relative holes 46, so that the side handle 20 for the application of the force can be positioned in the desired direction with respect to the metal-back. The body 40 and the element 42 are also connected by an internal spring 54 (FIG. 7), so that the main stem 12 acts as a single piece.

The central element 42 and the lower element 28 of the main stem 12 are connected with each other for example by means of a peg 48, which extends downwards, in an axial direction, from the central element 42 and which is inserted in a corresponding blind hole 50, axially positioned in the lower element 28. The torsional dynamometer 14, which forms the system for the application and measurement of the torsional moment on the implanted metal-back and which is produced, for example, in the form of a torsion spring, is therefore made integral with the central element 42 and lower element 28, in correspondence with their reciprocal contact portions.

The side handle 20 enables the application of a rotation to the upper body 40 of the main stem 12 and the central element 42 connected thereto by insertion, as far as the area in which there is the torsion spring 14. The spring 14 therefore transmits said rotation to the lower element 28 which, in turn, exerts it on the metal-back on which it has been previously screwed. The same torsion spring 14, suitably calibrated, allows the measurement of the torsional moment applied, consequently forming a torsional dynamometer.

The contemporaneousness of the two mechanical actions, i.e. destabilizing force and torsional moment, exerted on the implanted metal-back is guaranteed by the fact that the destabilizing force and the torsional moment are both applied by means of the side handle 20. By acting on the latter, the surgeon can contemporaneously exert a traction on the axial dynamometer 22 and a torsion on the torsional dynamometer 14. The reliability of the measurement of the two mechanical actions on the implanted metal-back is guaranteed by the accuracy of the calibration of the axial 22 and torsional 14 dynamometers.

Sterilizability and surgical safety are ensured by the fact that the surgical instrument 10 according to the present invention can be entirely made of stainless steel, like most of the surgical instruments on the market, or with an analogous hard and shock-resistant material. The possibility of applying the protective hood 38, made with a fine layer of polymeric material, on the free end of the secondary stem 18 allows the internal edge of the metal-back to be protected during the application of the destabilizing force, enabling the transmission of the force but excluding impact between the two metallic parts in contact.

The simplicity of the implantation procedure of the metal-back is maintained due to the fact that the surgical instrument 10 is essentially a variant of an impactor of the traditional type. The surgical time is lengthened only for verifying the stability on the implanted component, which in any case is quite simple and rapid. The simplicity and convenience of use of the surgical instrument 10 are also ensured by the fact that this is essentially a variant of the traditional impactor. The same verification of stability on the implanted component is in any case a simple and intuitive process.

The surgical instrument 10 according to the present invention does not require further instruments for exerting its function as the systems for the application and measurement of the mechanical actions on the implanted metal-back are integrated in the impactor.

The reusability of the surgical instrument 10, in which the pull and torsion springs, suitable calibrated for functioning as dynamometers, can also be made of stainless steel, is guaranteed by its sterilizability.

Finally, the possibility of simulating the mechanical stress actions on the acetabular component allows the implantation process to be verified, providing two important consequences:

on a clinical level, the surgeon can decide whether or not to use further fixing methods of the acetabular component by means of an objective test, in the intra-operative phase, without having to wait for the result a posteriori of the intervention (which would mean a re-intervention in the case of instability of the implant);

on a medical-legal level, the surgeon can indicate the verifications of the simulations in the clinical report, as a protection against possible recriminations on the part of the patient.

It can thus be seen that the surgical instrument for the implantation of hip prostheses according to the present invention, achieves the purposes specified above.

The surgical instrument for the implanting of hip prostheses according to the present invention thus conceived can in any case undergo numerous modifications and variants, all included in the same inventive concept; furthermore, all the details can be substituted by technically equivalent elements. In practice, the materials used, as also the forms and dimensions, can vary according to technical requirements.

The protection scope of the invention is therefore defined by the enclosed claims.

1. A surgical instrument (10) for the implantation of acetabular components of hip prostheses comprising:
a main stem (12), which acts as a dynamometric impactor for obtaining the implantation of said acetabular component and which comprises at least one torsional dynamometer (14), coaxial to said main stem (12), for the application and measuring of a torsional moment on said acetabular component implanted; and
a side mechanism (16), which comprises a secondary-stem (18), parallel to said main stem (12), a handle (20) and at least one axial dynamometer (22), orthogonal to said stem (18), which connects said secondary stem (18) to said handle (20) for the application and measuring of a destabilizing force on said acetabular component implanted.

2. The surgical instrument (10) according to claim 1, characterized in that said main stem (12) consists of an upper tubular body (40), at least one central element (42) coaxial to said upper tubular body (40), connected thereto by insertion, and at least one lower element (28), coaxial to said upper tubular body (40) and to said central element (42) and which can be rotated with respect to said at least one central element (42) by the interpositioning of said at least one torsional dynamometer (14).

3. The surgical instrument (10) according to claim 2, characterized in that said at least one lower element (28) is equipped with a threaded terminal end (30) for connection with said acetabular component.

4. The surgical instrument (10) according to claim 3, characterized in that said secondary stem (18) is equipped with a lower free end (32) which, together with said threaded terminal end (30) of said lower element (28), forms the interface system with said acetabular component.

5. The surgical instrument (10) according to claim 4, characterized in that said secondary stem (18) is capable of sliding parallel to said main stem (12) to adapt said interface system with said acetabular component to acetabular shells having different dimensions.

6. The surgical instrument (10) according to claim 4, characterized in that said lower free end (32) of said secondary stem (18) is covered with a protective hood (38) produced with a thin layer of polymeric material.

7. The surgical instrument (10) according to claim 2, characterized in that said side mechanism (16) is connected to said upper body (40) of said main stem (12) by means of one or more pass-through holes (24a, 24b), situated on said upper body (40) and having an axis perpendicular to the axis of said main stem (12), one or more pins (26a, 26b) obtained on said side mechanism (16) being respectively inserted, by sliding, into said holes (24a, 24b).

8. The surgical instrument (10) according to claim 7, characterized in that said secondary stem (18) is made integral with one or more sleeves (34a, 34b) in turn integral and coaxial respectively with said one or more pins (26a, 26b).

9. The surgical instrument (10) according to claim 8, characterized in that one or more end portions (36a, 36b) of said sleeve (20) are capable of sliding in said one or more sleeves (34a, 34b), respectively.

10. The surgical instrument (10) according to claim 2, characterized in that, in correspondence with the reciprocal interface portions, said upper body (40) is equipped with at least one pin (44) and said central element (42) has a plurality of axial holes (46) arranged along a circumference, said upper body (40) being able to be connected to said central element (42) according to different rotation degrees, by the insertion of said at least one pin (44) in one of said plurality of holes (46), so that said handle (20) can be positioned in the desired direction with respect to said acetabular component.

11. The surgical instrument (10) according to claim 10, characterized in that said upper body (40) and said central element (42) are connected by an internal spring (54).

12. The surgical instrument (10) according to claim 2, characterized in that said central element (42) and said lower element (28) are connected with each other by means of a peg (48) which extends downwards, in an axial direction, from said central element (42) and which is inserted in a corresponding blind hole (50) positioned axially in said lower element (28).

13. The surgical instrument (10) according to claim 1, characterized in that the sum between the radius of said main stem (12) and the diameter of said secondary stem (18) must not be greater than the internal radius of said acetabular component.

14. The surgical instrument (10) according to claim 1, characterized in that said torsional dynamometer (14) consists of a torsion spring.

15. The surgical instrument (10) according to claim 1, characterized in that said axial dynamometer (22) consists of a pull spring.

16. The surgical instrument (10) according to claim 1, characterized in that on the upper free end of said main stem (12) there is an impact component (52) suitable for receiving impact force to be transmitted to said acetabular component 36.

17. (canceled)
